(19) United States
(12) Patent Application Publication
(10) Pub. No.: US 2026/0371092 A1
(43) Pub. Date: Nov. 19, 2026

(54) METHODS, APPARATUS, AND SYSTEM FOR PAIRING A PATIENT'S MOBILE DEVICE WITH A MEDICAL DEVICE

(71) Applicants: VANTIVE US HEALTHCARE LLC, Deerfield, IL (US); VANTIVE HEALTH GMBH, Glattpark (Opfikon) (CH)

(72) Inventor: Mauro GUSELLA, Castlelvetro (IT)

(21) Appl. No.: 19/144,220

(22) PCT Filed: Dec. 20, 2023
(86) PCT No.: PCT/EP2023/086908

§ 371 Date: Jun. 27, 2025

(30) Foreign Application Priority Data
Dec. 27, 2022 (IT) 102022000026862

(51) Int. Cl. H04W 12/50; G16H 40/67; H04L 9/40; H04W 12/069

(52) U.S. Cl. CPC H04W 12/50; G16H 40/67; H04L 63/0869; H04W 12/069

(57) ABSTRACT

Methods, apparatus, and systems for pairing a patient's mobile device with a medical device are disclosed. The methods, systems, and apparatus use a server that is securely connected to a medical device to manage a pairing process. The secure connection between the server and the medical device is used to transmit a certificate in addition to a phone number of a patient's mobile device. The server also transmits a pairing code to the medical device using the secure connection. The medical device displays the pairing code. A patient causes the mobile device to record the code, which is transmitted to the server to perform a congruency check by comparing the received code to the generated code. When the codes match, the server transmits a device identifier of the mobile device to the medical device, which enables the medical device to establish a secure proximity communication channel with the medical device.

### BACKGROUND

Due to various causes, a person's renal system can fail. Renal failure produces several physiological derangements. For instance, it is no longer possible for a person with renal failure to balance water and minerals or to excrete daily metabolic load. Additionally, toxic end products of metabolism, such as, urea, creatinine, uric acid and others, may accumulate in a patient's blood and tissue.

Reduced kidney function and, above all, kidney failure is treated with dialysis. Dialysis removes waste, toxins and excess water from a patient's body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across a semi-permeable dialyzer between the blood and an electrolyte solution, called dialysate or dialysis fluid, to cause diffusion. The diffusion occurs externally from the patient, where an extracorporeal circuit is used for removing uncleansed blood and returning cleansed blood to the patient.

Hemofiltration (“HF”) is an alternative renal replacement therapy that relies on a convective transport of toxins from a patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large toxic molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Another type of kidney failure therapy is peritoneal dialysis (“PD”), which infuses a dialysis solution, also called dialysis fluid, into a patient's peritoneal chamber via a catheter. The dialysis fluid is in contact with the peritoneal membrane in the patient's peritoneal chamber. Waste, toxins and excess water pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins and excess water from the patient. This cycle is repeated multiple times.

There are various types of peritoneal dialysis therapies, including continuous ambulatory peritoneal dialysis (“CAPD”), automated peritoneal dialysis (“APD”), tidal flow dialysis, and continuous flow peritoneal dialysis (“CFPD”). CAPD is a manual dialysis treatment. Here, the patient manually connects an implanted catheter to a drain to allow used or spent dialysis fluid to drain from the peritoneal chamber. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal chamber, where the transfer of waste, toxins and excess water takes place. After a dwell period, the patient repeats the manual dialysis procedure, for example, four times per day. Manual peritoneal dialysis requires a significant amount of time and effort from the patient, leaving ample room for improvement.

Automated peritoneal dialysis (“APD”) is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh dialysis fluid and to a fluid drain. APD machines pump fresh dialysis fluid from a dialysis fluid source, through the catheter and into the patient's peritoneal chamber. APD machines also allow for the dialysis fluid to dwell within the chamber and for the transfer of waste, toxins and excess water to take place. The source may include multiple liters of dialysis fluid including several solution bags.

APD machines pump used or spent dialysate from the patient's peritoneal cavity, though the catheter, and to the drain. As with the manual process, several drain, fill and dwell cycles occur during dialysis. A “last fill” may occur at the end of the APD treatment. The last fill fluid may remain in the peritoneal chamber of the patient until the start of the next treatment, or may be manually emptied at some point during the day.

In any of the above modalities, the automated dialysis machine may be located in a patient's home. In some instances, the automated dialysis machine permits a patient to select which dialysis prescription, among a plurality of stored prescriptions, is performed for a next treatment. Alternatively, the automated dialysis machine may allow a patient to change certain prescription parameters, such as a fill volume or dwell time for PD. The ability for a patient to control their dialysis treatment helps them stay engaged and improves treatment compliance.

To improve patient engagement with their dialysis treatment, some known automated dialysis machines receive inputs from a patient using a dialysis application operating on a smartphone or tablet. While the use of a smartphone or tablet makes it easier for a patient to manage their treatments, it is often difficult for the patient to provision their smartphone or tablet to communicate with the automated dialysis machine. Patients receiving dialysis treatments are typically older and not as well versed in pairing devices, especially pairing procedures that may require many complex steps or require connecting to a remote server. Further, to the extent pairing can be accomplished using a single username and password, some legal jurisdictions, such as the European Union, require two-factor authentication to safeguard sensitive medical data.

A need accordingly exists for a dialysis system that enables a patient to effortlessly pair a mobile device with a medical device in compliance with jurisdictions that require at least two-factor authentication.

### SUMMARY

Example systems, methods, and apparatus are disclosed herein that pair a patient's mobile device with a medical device, such as an automated dialysis machine. The systems, methods, and apparatus leverage a secure connection between a medical device and a remote or cloud-based server. This secure connection enables certain data (e.g., a phone number of a patient's mobile device) and certificates to be securely communicated between the server and the medical device. The server uses the medical device for providing information, such as a pairing code, to enable the patient to authenticate the mobile device. The patient enters the pairing code or records an image of the pairing code, which is transmitted from the mobile device to the server to perform a congruency check to confirm the mobile device is enabled to communicate with the medical device. The congruency check may include comparing the pairing code received from the mobile device to the pairing code transmitted from the server to the medical device to ensure the pairing codes match. The congruency check may also include ensuring that a clinician of the patient authorized or enabled the patient to communicate with the medical device.

After the congruency check is complete, the server transmits a message including an International Mobile Equipment Identity (“IMEI”) number or other identifier of the mobile device to the medical device. The message may also indicate that the medical device is approved for pairing with the mobile device. After receiving the message, the medical device is configured to open a proximity communication channel with the mobile device. The proximity communication channel may include a Bluetooth® channel, a Wi-Fi direct channel, a local Wi-Fi channel, a near-field communication (“NFC”) channel, a Zigbee® channel, a LoRa channel, a Z-Wave® channel, a WeMo® channel, or a low-power wide-area network (“LPWAN”) channel. In some instances, the server also transmits a message to the mobile device to open the proximity communication channel to enable pairing with the medical device.

To pair, the medical device uses the IMEI number and certificate received from the server to establish a secure proximity communication channel with the mobile device. The mobile device may then use the certificate to communicate in a secure and trusted manner with the medical device over a local wireless connection. Such a connection enables the mobile device to communicate directly with the medical device without having to route communications through the server over a public network. Further, to pair, the patient only needed to record a pairing code displayed by the medical device, which enables relatively less technologically savvy patients to easily and quickly pair their mobile device with their medical device.

In light of the disclosure herein and without limiting the disclosure in any way, in a first aspect of the present disclosure, which may be combined with any other aspect listed herein, a control arrangement to enable medical device interoperability includes a system for pairing a patient device with a medical device includes a medical device configured to perform a treatment or measurement on a patient. The medical device is communicatively coupled to a network. The system also includes a database configured to correlate a patient phone number and a device identifier with an identifier, an electronic address, or a device identifier of the medical device. The system further includes a server communicatively coupled to the database and the network. The server is securely connected to the medical device via a mutual authentication mechanism. The server is configured to generate a patient certificate and transmit the patient certificate and the patient phone number to the medical device to enable pairing. The server is also configured to generate a pairing code after receiving a code request message from the medical device, store to the database an association between the pairing code and the patient phone number, and transmit the pairing code to the medical device, causing a display screen of the medical device to display the pairing code. The system moreover includes a patient device communicatively coupled to the network. The patient device includes a processor and a memory device storing one or more instructions defining an application for communicating with the medical device, execution of the one or more instructions by the processor causing the application on the patient device to at least receive a selection of a pairing input, after receiving the pairing input, enable the patient device to record information indicative of the pairing code displayed by the medical device, transmit the pairing code and the patient phone number to the server causing the server to perform a congruency check by comparing the received pairing code and the patient phone number to the pairing code and the patient phone number stored in the database, and when there is a match between the received pairing code and the patient phone number and the pairing code and the patient phone number stored in the database, receive from the server a pairing acceptance message that causes the patient device to open a proximity communication channel. In addition to above, the server is configured to transmit the pairing acceptable message and the device identifier of the patient device to the medical device when there is a match between the received pairing code and the patient phone number and the pairing code and the patient phone number stored in the database. Further, the medical device is configured to open the proximity communication channel using the patient phone number and the device identifier to communicate with the patient device. Additionally, the medical device transmits the patient certificate to the application of the patient device using the opened communication channel, enabling the application to communicate with the medical device and the server via the network.

In a second aspect of the present disclosure, which may be combined with any other aspect listed herein, the server is configured to receive from a clinician device a patient device enable message to permit the patient to pair the patient device using the application.

In a third aspect of the present disclosure, which may be combined with any other aspect listed herein, wherein, after receiving the patient device enable message, the server is configured to generate patient certificate.

In a fourth aspect of the present disclosure, which may be combined with any other aspect listed herein, the server additionally causes the application to enable selection of the pairing input after receiving the patient device enable message.

In a fifth aspect of the present disclosure, which may be combined with any other aspect listed herein, the database receives at least one of the patient phone number or the device identifier from the clinician device via the patient device enable message.

In a sixth aspect of the present disclosure, which may be combined with any other aspect listed herein, the application on the patient device is further configured to transmit a pairing completed message to the server, enabling the server to display an indication to the clinician device indicative of the pairing of the patient device.

In a seventh aspect of the present disclosure, which may be combined with any other aspect listed herein, the application is configured to transmit at least one of the patient phone number or the device identifier to the database after the application is installed on the patient device.

In an eight aspect of the present disclosure, which may be combined with any other aspect listed herein, the application is configured to transmit the device identifier of the patient device after receiving the pairing acceptance message from the server.

In a ninth aspect of the present disclosure, which may be combined with any other aspect listed herein, the patient certificate includes a parts manufacturer approval (“PMA”) certificate.

In a tenth aspect of the present disclosure, which may be combined with any other aspect listed herein, the device identifier includes at least one of a media access control (“MAC”) address or an International Mobile Equipment Identity (“IMEI”) number.

In an eleventh aspect of the present disclosure, which may be combined with any other aspect listed herein, the proximity communication channel encompasses at least one of a Bluetooth® protocol, a Wi-Fi direct protocol, a local Wi-Fi protocol, a near-field communication (“NFC”) protocol, a Zigbee® protocol, a LoRa protocol, a Z-Wave® protocol, a WeMo® protocol, or a low-power wide-area network (“LPWAN”) protocol, and wherein the network includes at least one of a cellular network or a wide area network.

In a twelfth aspect of the present disclosure, which may be combined with any other aspect listed herein, the medical device includes a user interface configured to receive a pairing request input, and wherein the medical device transmits the code request message to the server after receiving the pairing request input.

In a thirteenth aspect of the present disclosure, which may be combined with any other aspect listed herein, the pairing code includes at least one of a Quick-Response (“QR”) code, a barcode, or an alpha-numeric code.

In a fourteenth aspect of the present disclosure, which may be combined with any other aspect listed herein, the database is configured to correlate the patient phone number or identifier based on a clinician request.

In a fifteenth aspect of the present disclosure, which may be combined with any other aspect listed herein, the server and the medical device each include a copy of a same certificate to communicate via the mutual authentication mechanism.

In a sixteenth aspect of the present disclosure, which may be combined with any other aspect listed herein, the patient device includes at least one of a cellular phone, a smartphone, a tablet computer, a smartwatch, a laptop computer, or a desktop computer.

In a seventeenth aspect of the present disclosure, which may be combined with any other aspect listed herein, the application on the patient device, the server, and the medical device are configured to use the patient certificate to encrypt communications therebetween.

In an eighteenth aspect of the present disclosure, which may be combined with any other aspect listed herein, the medical device includes at least one of an automated peritoneal dialysis machine, a hemodialysis machine, a continuous renal replacement therapy (“CRRT”) machine, an infusion pump, a perinatal nutrition machine, a patient-controlled analgesia (“PCA”) pump, a water purification machine, a nutrition compounding machine, a bedside monitor, an alarm monitoring/control station, a pulse oximeter, a weight scale, a heart rate monitor, an ECG monitor, a thermometer, or a pressure sensor.

In a nineteenth aspect of the present disclosure, which may be combined with any other aspect listed herein, the server also performs the congruency check by confirming a clinician of the patient provided an indication that the patient is permitted to pair the patient device with the medical device.

In a twentieth aspect of the present disclosure, which may be combined with any other aspect listed herein, the information indicative of the pairing code is obtained via an image recorded by a camera of the patient device

In a twenty-first aspect of the present disclosure, which may be combined with any other aspect listed herein, the disclosure relates to an apparatus for pairing a patient device with a medical device. The apparatus comprising a database configured to correlate a patient phone number and a device identifier with an identifier, an electronic address, or a device identifier of a medical device that is configured to perform a treatment or a measurement on a patient. The apparatus also comprises a server communicatively coupled to the database and securely connected to the medical device via a mutual authentication mechanism over a network. The server is configured to generate a patient certificate and transmit the patient certificate and the patient phone number to the medical device to enable pairing. The server is also configured to generate a pairing code after receiving a code request message from the medical device and to store to the database an association between the pairing code and the patient phone number. The server is further configured to transmit the pairing code to the medical device, enabling a display screen of the medical device to display the pairing code. The server is also configured to receive, from an application operating on a patient device communicatively coupled to the network, the pairing code and the patient phone number as deciphered from an image recorded by a camera of the patient device of the display screen of the medical device and perform a congruency check by comparing the received pairing code and the patient phone number to the pairing code and the patient phone number stored in the database. Further, the server is configured to when there is a match between the received pairing code and the patient phone number and the pairing code and the patient phone number stored in the database: transmit to the patient device a pairing acceptance message that causes the patient device to open a proximity communication channel, and transmit the pairing acceptable message and the device identifier of the patient device to the medical device, causing the medical device to open the proximity communication channel using the patient phone number and the device identifier to communicate with the patient device, wherein the medical device transmits the patient certificate to the application of the patient device using the opened communication channel, enabling the application to communicate with the medical device and the server via the network.

In a twenty-second aspect of the present disclosure, which may be combined with any other aspect listed herein, the disclosure relates to a medical device apparatus for pairing with a patient device. The medical device apparatus comprises: a memory device configured to store at least a pairing code, a patient phone number, a device identifier, and a patient certificate. The apparatus further comprises a user interface; a display screen and a processor. The processor is communicatively coupled to the memory device, the display screen, and the user interface. The processor and memory device are configured to cooperatively: receive, from a server via a network, the patient certificate and the patient phone number, wherein the processor is authenticated to communicate with the server, to store, to the memory device, the patient certificate and the patient phone number, to receive, via the user interface, a request to pair with a patient device, to transmit, to the server, a request for a pairing code, to receive, from the server, the pairing code, to display, via the display screen, the pairing code to enable a camera of the patient device to record an image of the pairing code, to receive, from the server, a pairing acceptable message and the device identifier of the patient device when there is a match between the pairing code and the patient phone number received in the server from the patient device and the pairing code and the patient phone number stored in a database, to open a proximity communication channel using the patient phone number and the device identifier to communicate with the patient device, and to transmit the patient certificate to the application of the patient device using the opened communication channel, enabling the processor to communicate with the application of the patient device.

In a twenty-third aspect of the present disclosure, which may be combined with any other aspect listed herein, the disclosure relates to a patient device apparatus for pairing with a medical device. The apparatus comprises: a display screen; a camera; a memory device storing one or more instructions defining an application for communicating with the medical device; and a processor communicatively coupled to the display screen, the camera, and the memory device. The processor is configured to execute the one or more instructions causing the application to: receive a selection of a pairing input; after receiving the pairing input, enable the camera to record an image of a pairing code displayed by the medical device; decipher the pairing code from the recorded medical image; transmit the pairing code and a patient phone number to a server via a network to cause the server to perform a congruency check by comparing the transmitted pairing code and the patient phone number to a pairing code and a patient phone number stored in a database, and, when there is a match between the transmitted pairing code and the patient phone number and the pairing code and the patient phone number stored in the database, receive from the server a pairing acceptance message; after receiving the pairing acceptance message, open a proximity communication channel; receive a patient certificate from the medical device via the opened communication channel, and use the patient certificate to enable communication with the medical device over the network.

In a twenty-fourth aspect of the present disclosure, any of the structure and functionality disclosed in connection with FIGS. 1 to 11B may be combined with any of the other structure and functionality disclosed in connection with FIGS. 1 to 11B.

In light of the present disclosure and the above aspects, it is therefore an advantage of the present disclosure to provide a system that enables a patient to easily and securely pair a medical device with a mobile device.

It is another advantage of the present disclosure to provide multi-factor authentication to enable a patient to pair a mobile device with a medical device to protect the transmission of medical data.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 shows a diagram of a medical system for pairing a medical device with a mobile device, according to an example embodiment of the present disclosure.

FIGS. 2 to 9 show an example process performed using the medical system of FIG. 1 for pairing the medical device with the mobile device, according to an example embodiment of the present disclosure.

FIG. 10 is a diagram of an example user interface provided by a patient software application of the mobile device showing a pairing of the mobile device to the medical device, according to an example embodiment of the present disclosure.

FIGS. 11A and 11B are flow diagrams of an example procedure for pairing a medical device with a mobile device, according to an example embodiment of the present disclosure.

### DETAILED DESCRIPTION

Methods, systems, and apparatus are disclosed for pairing a patient's mobile device with a medical device. The methods, systems, and apparatus use a server that is securely connected to a medical device to manage the pairing process. The secure connection between the server and the medical device is used to transmit at least one certificate, such as an X.509 certificate or a parts manufacturer approval (“PMA”) certificate, to a medical device in addition to a phone number of a mobile device for a patient. The server also transmits a pairing code to the medical device using the secure connection. The medical device displays the pairing code, which may be shown as a quick-response (“QR”) code, a barcode, alphanumeric characters, etc. A patient causes the mobile device to record the code. For example, a patient may use the mobile device to record a picture of the code, which is then translated into alphanumeric characters and transmitted to the server. In another example, an application on the mobile device may prompt a patient to enter the pairing code displayed as alphanumeric characters by the medical device.

The server receives the pairing code from the mobile device via at least one message. The server may also determine or receive a phone number or other identifier associated with the mobile device. The server then performs a congruency check by comparing the pairing code received from the mobile device to the pairing code generated and transmitted to the medical device. When the codes match, the server is configured to transmit an IMEI number of the mobile device to the medical device. The server may additionally perform a congruency check by ensuring that a clinician authorized the mobile device to communicate with the medical device. For instance, a clinician may create a record with pairing authorization that is stored to a patient's electronic medical record (“EMR”) or a data structure that correlates patients, phone numbers, mobile device identifiers, medical device identifiers, and indications as to which patients are permitted to pair with their medical devices.

Reception of the IMEI number by the medical device causes the medical device to open a proximity communication channel with the mobile device. In some instances, the server transmits an acceptance message including the IMEI number. The message includes information indicative that the medical device should open the proximity communication channel with the mobile device. Further, in some instances, the server may transmit a message to the mobile device that causes the mobile device to communicate with the medical device using the proximity communication channel. After the channel is open, the medical device is configured to transmit the certificate to the mobile device to complete the pairing process. The mobile device thereafter uses the certificate to securely communicate with the medical device using the proximity communication channel.

A patient may use the proximity communication channel to select a dialysis prescription and/or select parameters for a dialysis prescription. The selected prescription or parameters are transmitted directly to the medical device without being transmitted over a public network. Additionally, the mobile device is configured to receive medical data from the medical device. The mobile device shows the medical data and/or trends of the medical data overtime to enable a patient to track the progress of their treatments. In some instances, the mobile device may use the medical data to display information indicative of patient compliance with the treatment, thereby keeping the patient engaged with the dialysis therapy.

As one can appreciate, the methods, system, and apparatus enable a patient to create a secure, local connection with a medical device simply by recording a pairing code that is displayed by the medical device. The ease of pairing enables virtually any patient regardless of their health, age, or familiarity with technology to pair a mobile device with a medical device without intervention from a clinician or support staff. Further, the secure communication of certificates helps safeguard the pairing process and protects the transmission of medical data. Additionally, since a local proximity communication channel is used, such as Wi-Fi, a WLAN, or Bluetooth®, medical data is further protected since it is not transmitted over a public network.

Reference is made herein to automated medical devices. It should be appreciated that the methods, systems, and apparatus may provide pairing between a mobile device and any type of medical device including a large-volume infusion pump, a syringe pump, a patient-controlled analgesia pump, a parenteral nutrition pump, a PD machine, an HD machine, a HDF machine, a CRRT machine, a ventilator, a physiological monitor/sensor, and/or a patient bedside monitor. Each of these medical devices includes a screen for displaying a pairing code and includes a capability to communicate over a proximity communication channel.

Reference is made herein to medical data. As disclosed, medical data is generated at a medical device and is available for transmission to a server or mobile device. The medical data includes treatment programming information. Treatment programming information includes one or more parameters that define how a medical device is to operate to administer a treatment to a patient. For a peritoneal dialysis therapy, the parameters may specify an amount (or rate) of fresh dialysis fluid to be pumped into a peritoneal cavity of a patient, an amount of time the fluid is to remain in the patient's peritoneal cavity (i.e., a dwell time), and an amount (or rate) of used dialysis fluid and ultrafiltration (“UF”) that is to be pumped or drained from the patient after the dwell period expires. For a treatment with multiple cycles, the parameters may specify the fill, dwell, and drain amounts for each cycle and the total number of cycles to be performed during the course of a treatment (where one treatment is provided per day or separate treatments are provided during the daytime and during nighttime). For CRRT, the parameter may include a continuous UF rate. In addition, the parameters may specify dates/times/days (e.g., a schedule) in which treatments are to be administered by the medical fluid delivery machine. Further, parameters of a prescribed therapy may specify a total volume of dialysis fluid to be administered for each treatment in addition to a concentration level of the dialysis fluid, such as a dextrose level. For an infusion therapy, the parameters may include a volume to be infused, a medication to be infused, a medication concentration, a medication dosage, and/or an infusion rate.

For the AK 98™ hemodialysis machine manufactured by Baxter International Inc., the treatment programming parameters may include a UF volume, a treatment time, a concentrate type, a sodium concentration, a bicarbonate concentration, a heparin bolus volume, a heparin flow rate, a heparin stop time, a heart rate monitoring flag, a dialysis fluid temperate, a dialysis fluid flow rate, and indications as to whether UF is isolated, UF profiling is to occur, a single needle is being used for arterial and venous connections, whether sodium profiling is to occur, whether bicarbonate profiling is to occur, and/or whether dialysis fluid conductivity monitoring is to occur. A blood flow rate to a dialyzer may also be a treatment programming parameter. For HDF, pre-and post-infusion volumes may also be treatment programming parameters.

The medical data also includes event information that relates to administration of the treatment. The event information may include data generated by a medical device that is indicative of measured, detected, or determined parameter values. For example, while a prescribed therapy may specify that a treatment is to comprise five separate cycles, each with a 45 minute dwell time, a medical fluid delivery device may administer a treatment where fewer cycles are provided, each with a 30 minute dwell time. The medical device monitors how the treatment is administered and accordingly provides parameters that are indicative of the operation. The parameters for the treatment data may include, for example, a total amount of dialysis fluid administered to the patient, a number of cycles operated, a fill amount per cycle, a dwell time per cycle, a drain time/amount per cycle, an estimated amount of UF removed, a treatment start time/date, and/or a treatment end time/date. The treatment data may also include calculated parameters, such as a fill rate and a drain rate, determined by dividing the amount of fluid pumped by the time spent pumping. The treatment/event data may further include an identification of an alarm that occurred during a treatment, a duration of the alarm, a time of the alarm, an event associated with the alarm, and/or an indication as to whether the issue that caused the alarm was resolved or whether the alarm was silenced.

The medical data further includes device machine logs that include diagnostic information, fault information, etc. The diagnostic information may include information indicative of internal operations of a medical device, such as faults related to pump operation, signal errors, communication errors, software issues, etc. The diagnostic information may also include setup information, such as steps performed to connect tubing to the medical device and prime/disinfect the tubing. The medical data may be transmitted as a data stream or provided at periodic intervals. In some instances, the medical data may be transmitted as events or other changes to the data occur.

### I. Medical System for Device Pairing Embodiment

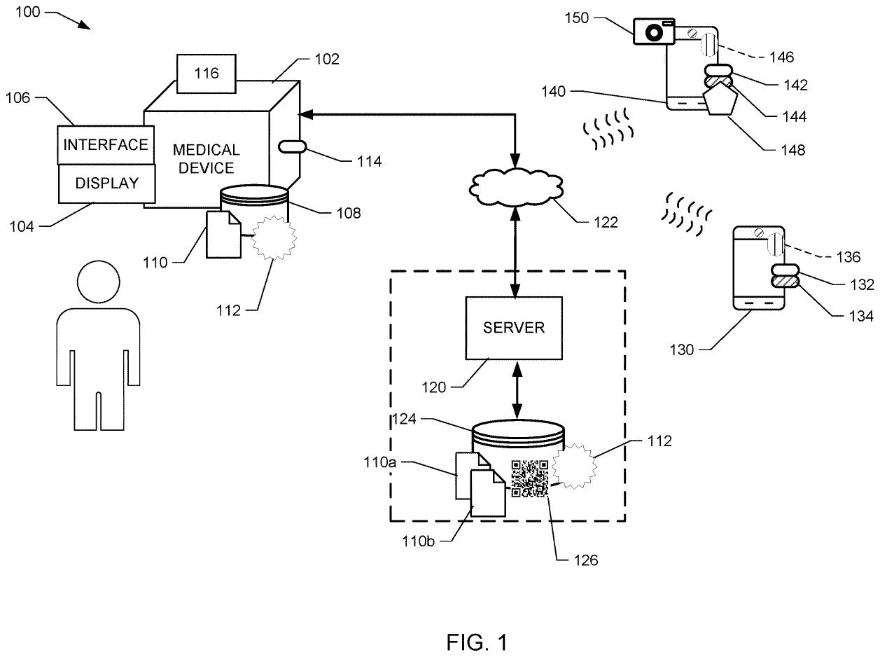

FIG. 1 shows a diagram of a medical system 100 for pairing a medical device with a mobile device, according to an example embodiment of the present disclosure. The example medical system 100 includes a medical device 102, which may be located in a patient's home. The medical device 102 may include the Amia Automated PD system manufactured by Baxter® International Inc. The medical device 102 may also be the PrisMax CRRT machine manufactured by Baxter International Inc. In other embodiments, the medical device 102 may include an intermediate hemodialysis machine, an infusion pump (e.g., a syringe pump, a linear peristaltic pump, a large volume pump (“LVP”), an ambulatory pump, multi-channel pump), a nutritional compounding machine, a water preparation machine, a dialysis fluid generation machine, a cycler, etc. The medical device 102 may also include a bedside patient monitor or physiological sensors such as an oxygen sensor, a respiratory monitor, a glucose meter, a concentration meter, a conductivity meter, a blood pressure monitor, an electrocardiogram (“ECG”) monitor, a weight scale, a heart rate monitor, or any other peripheral medical device configured to sense a physiological parameter of a patient or of produced fluid. While FIG. 1 shows a single medical device 102, it some embodiments a patient may pair two or more medical devices with a mobile device. Further, it should be appreciated that the system 100 provides for the pairing of thousands of medical devices with respective mobile devices.

The medical device 102 is configured to accept one or more parameters specifying a treatment or prescription (i.e., treatment programming information). During operation, the medical device 102 generates event, diagnostic, and/or operational data (e.g., medical data). In some embodiments, the medical data conforms to the ISO/IEEE 11073™ standards as XML-based information objects. In other embodiments, the medical data is in a different format, such as JavaScript Object Notation (“JSON”), a HyperText Markup Language (“HTML”), a comma-separated values (“CSV”), text, and/or Health-Level-7 (“HL7”).

In the illustrated example, the medical device 102 includes a display device 104 that shows one or more screens providing a graphical representation of medical data, and more generally a status of a patient treatment or setup. The display device 104 may include a touchscreen that is configured to receive inputs. Additionally or alternatively, the display device 104 may operate in connection with a control interface 106 that enables a patient to select options shown on the screen. The control interface 106 may include buttons, a control panel, or the touchscreen of the display device 104. The control interface 106 may also be configured to enable a patient to navigate to a certain screen for display. The control interface 106 may also provide instructions for operating or controlling the medical device.

The medical device 102 also includes a memory device 108 that is configured to store one or more certificates 110, a medical device identifier 112, a phone number of a mobile device, and/or an EMEI of the mobile device. The memory device 108 may also store prescriptions, treatment parameters, and medical data. As discussed below, the certificates 110 enable secure communication between the medical device 102 and a mobile device. The medical device identifier 112 may include a serial number, destination address, etc. that is used to identify and/or address communications to the medical device 102. The memory device 108 may include any RAM, ROM, EEPROM, flash drive, solid state drive, distributed database, etc.

The medical device 102 further includes a processor 114 that is communicatively coupled to the display device 104, the control interface 106, and the memory device 108. The processor 114 is configured to generate and/or process medical data, which is stored in the memory device 108. The processor 114 may generate and process the medical data in a HL7 format, an XML format, a binary version 2 format, a binary version 3 format, or a Fast Healthcare Interoperability Resources (“FHIR”) format. As discussed below, the processor 114 is also configured to pair the medical device 102 with at least one mobile device.

The medical data for the medical device 102 may include a UF volume, a treatment time, a concentrate type, a sodium concentration, a bicarbonate concentration, a heparin bolus volume, a heparin flow rate, a heparin stop time, a heart rate monitoring flag, a dialysis fluid temperate, a dialysis fluid flow rate, and indications as to whether UF is isolated, UF profiling is to occur, a single needle is being used for arterial and venous connections, whether sodium profiling is to occur, whether bicarbonate profiling is to occur, and/or whether dialysis fluid conductivity monitoring is to occur. The medical data may also include a blood flow rate to a dialyzer and pre- and post-infusion volumes. Further, the medical data may include events and/or alarms, such as priming information, line connection/disconnection information, disinfection/cleaning information, occlusion detections, significant pressure or flow rate fluctuations or changes, etc. The processor 114 creates medical data in conjunction with operating one or more pumps or other components to administer the treatment. The medical data may further include a prescription value for a total treatment time during which a patient is to be submitted to a blood treatment, a prescription value for a total patient fluid removal to be achieved by an end of a total treatment time, and a prescription value for an average patient fluid removal rate to be kept across the total treatment time.

In some embodiments, one or more physiological sensors may be communicatively coupled to one of the medical device 102. For instance, FIG. 1 shows a physiological sensor 116 connected to the home-based medical device 102. The sensor 116 may include an oxygen sensor, a respiratory monitor, a glucose meter, a blood pressure monitor, an electrocardiogram (“ECG”) monitor, a weight scale, a heart rate monitor, etc. The processor 114 of the medical device 102 may be configured to include data from the physiological sensors as the medical data that is transmitted to a mobile device.

The processor 114 of the medical device 102 operates according to one or more instructions for performing a treatment on a patient. The instructions may be acquired via the control interface 106 or via the memory device 108. The processor 114 also monitors devices components for issues, which are documented as diagnostic medical data. While the medical device 102 is shown as having one processor 114, in some embodiments, the medical device 102 may include two or more processors 114 with specified operations.

As shown in FIG. 1, the medical device 102 is communicatively coupled to a server 120 via a network 122. For the home-based medical device 102, the network connection may include any combination of an Ethernet connection, an Internet connection, Wi-Fi connection, a wireless local area network (“WLAN”), and/or a cellular 5G/6G connection. The network 122 may include one or more of an access point, a router, a switch, a repeater, or other telecommunication equipment for routing communications in a network.

The medical device 102 may have a secure communication connection with the server 120. For example, the medical device 102 and the server 120 may include X.509 certificates or PMA certificates for secure communication. Additionally or alternatively, the medical device 102 and the server 120 may include encryption keys to encrypt/decrypt communications therebetween. Further, the medical device 102 may communicate with the server 120 via a virtual network connection (“VNC”).

In the illustrated example, the server 120 is configured to store the certificates 110 to a memory device or database 124. The server 120 is also configured to store medical device identifiers 112 and generated pairing codes 126 to the memory device 124. The server 120 may also store to the memory device 124 a phone number of a patient's mobile device, an EMEI of the mobile device, and/or an approval from a clinician indicating that the patient is permitted to pair their mobile device with an assigned medical device. In some embodiments, the identifiers and pairing authorization is stored to an EMR of the patient within the memory device 124. Additionally or alternatively, the identifiers and pairing authorization is stored to a file, database, or other data structure configured to manage pairing information. For example, a database may correlate the medical device identifier 112, the certificate 110, a patient identifier, a phone number of a patient's mobile device, an EMEI of a patient's mobile device, a pairing code, and/or an indication of pairing approval from a clinician.

The example server 120 may be provisioned in a cloud-based network. Alternatively, the server 120 may be hosted within a medical network, such as a hospital information system. Regardless of the location, the server 120 is configured to manage the pairing process between medical devices and patient mobile devices, as discussed in more detail below.

The medical system 100 includes a clinician device 130 for enabling a clinician to authorize a patient to pair a mobile device with a medical device. The clinician device 130 may include a smartphone, a tablet computer, a laptop computer, a desktop computer, a workstation, a clinician station, etc. While FIG. 1 shows a single clinician device 130, it should be appreciated that the medical system 100 may include a plurality of clinician devices that are connected to the server 122 via the local or wide area network 122.

The clinician device 130 includes a processor 132 and a memory device 134. Instructions are stored on the memory device 142. Execution of the instructions by the processor 132 cause the processor 132 to operate a software application 136 to perform the operations described herein. The processor 132 may comprise digital and/or analog circuity structured as a microprocessor, application specific integrated circuit (“ASIC”), controller, etc. The memory device 134 includes a volatile or non-volatile storage medium. Further, the memory device 134 may include any solid state or disk storage medium.

The software application 136 may authorize the clinician device 130 to communicate with the server 120. Additionally or alternatively, the software application 136 may include a login to ensure only authenticated users may access the server 120. After authentication, the software application 136 establishes a secure connection with the server 120, as discussed in more detail below.

The software application 136 is configured to display one or more user interfaces to enable a clinician to authorize a patient to pair a mobile device with a medical device. To enable the authorization, the software application 136 may prompt the clinician for a patient identifier. In response, the software application 136 transmits the patient identifier to the server 120, which uses the EMRs and/or databases stored at the memory device 124 to determine the patient identifier is associated with the medical device identifier 112 of the medical device 102 (and any other associated medical devices). The server 120 transmits to the software application 136 a list of the associated medical devices. A clinician then uses the software application 136 to select the medical device to indicate the patient is authorized to pair a mobile device with the medical device. In some embodiments, only one medical device may be associated with a patient such that the server 120 only provides an identifier of this medical device when requested. In some instances, the EMR of the patient or the database already has a mobile device number and/or EMEI as part of a patient registration process. Alternatively, the software application 136 is configured to prompt the clinician for a phone number of the patient's mobile device, which is transmitted to the server 120 and stored to the memory device 124.

The medical system 100 also includes a patient mobile device 140 for pairing with a medical device. The patient mobile device 140 may include a smartphone, a tablet computer, a laptop computer, a desktop computer, a workstation, a clinician station, etc. While FIG. 1 shows a single patient mobile device 140, it should be appreciated that the medical system 100 may include a plurality of patient mobile devices that can be paired with one or more medical devices.

The patient mobile device 140 includes a processor 142 and a memory device 144. Instructions are stored on the memory device 144. Execution of the instructions by the processor 142 cause the processor 142 to operate a patient software application 146 to perform the operations described herein. The processor 142 may comprise digital and/or analog circuity structured as a microprocessor, application specific integrated circuit (“ASIC”), controller, etc. The memory device 144 includes a volatile or non-volatile storage medium. Further, the memory device 144 may include any solid state or disk storage medium.

The patient software application 146 is configured to enable a patient to pair the mobile device 140 with the medical device 102. To enable pairing as discussed herein, the patient mobile device 140 is configured to store a device identifier 148, such as an EMEI, to the memory device 144. Alternatively, the device identifier 148 may hardcoded into the processor 142 or another hardware component of the mobile device 140. The patient software application 146 may transmit the device identifier 148 to the server 120 for storage in association with a patient identifier, a phone number of the mobile device 140, and corresponding medical data.

The mobile device 140 also includes a camera 150. In some embodiments, the display 104 of the medical device 102 shows a pairing code as a QR or barcode. In these embodiments, the camera 150 is used to record one or more images of the pairing code, which is then converted into corresponding alphanumeric characters by the processor 142. In other instances, the pairing code is displayed as alphanumeric characters, which may be entered by a patient into the patient software application 146. In these instances, the camera 150 may be omitted.

In some embodiments, the software application 146 is configured to prompt a patient for a code and/or biometric information during registration. The code, such as a 6-digit code, and the biometric information may be used periodically by the software application 146 to authenticate the user at the mobile device 140. In some instances, the software application 146 is configured to prompt the patient to enter the code or biometric information to initiate the pairing process or swap an already paired medical device.

### II. Device Pairing Method Embodiment

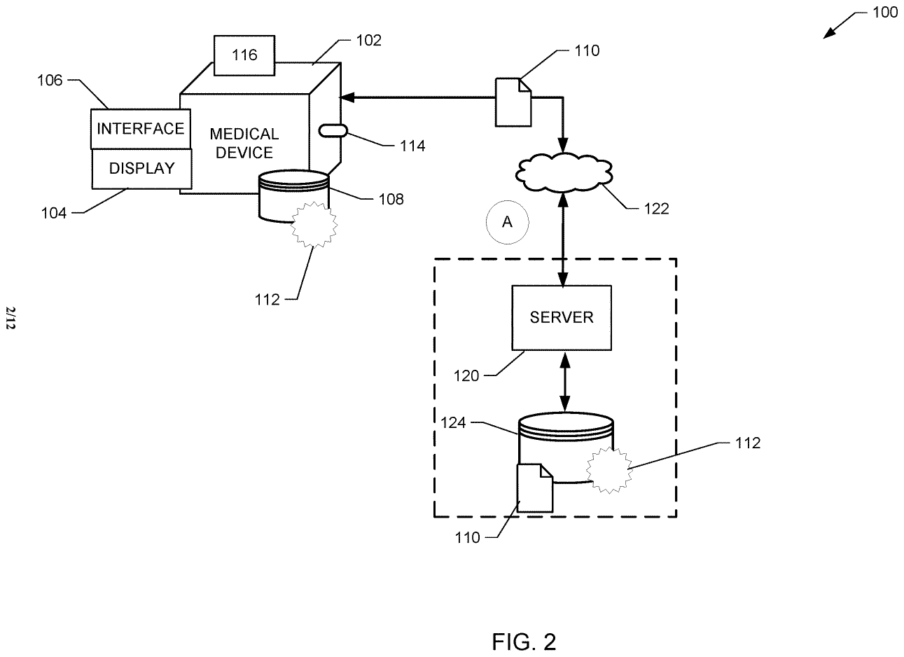

FIGS. 2 to 9 are diagrams illustrative of an example process performed by the medical system 100 of FIG. 1 to pair the medical device 102 with the patient mobile device 140, according to an example embodiment of the present disclosure. At Event A in FIG. 2, the medical device 102 and server 120 create a secure communication connection via the network 122. As part of this process, the server 120 may transmit a certificate 110 to the medical device 102 to enable the server 120 to securely communicate with the medical device 102 using the certificate to validate the communications (e.g., a mutual authentication mechanism). Alternatively, the certificate 110 may be replaced with one or more encryption keys. In yet other instances, the server 120 may establish a VNC or a VPN connection with the medical device 102. In this embodiment, an electronic address and/or an identifier 112 of the medical device 102 is stored by the server 120 to the memory device 124. The server 120 is configured to associate the certificate 110 with the electronic address and/or an identifier 112.

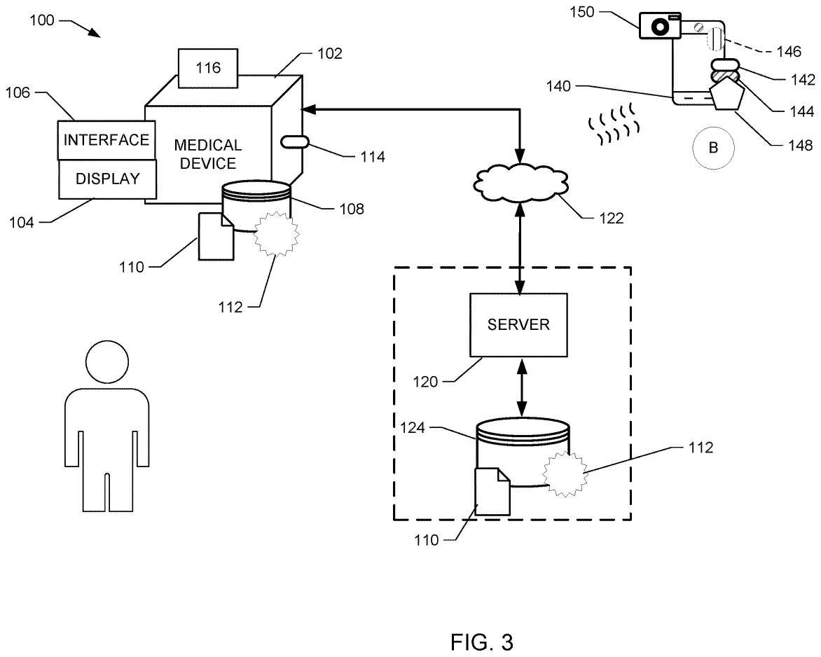

At Event B in FIG. 3, the patient may register with the server 120. Alternatively, a clinician may register the patient. Registration may include storing a phone number and/or device identifier 148 at the memory device 124. Registration may also include storing a patient identifier in association with the certificate 110 with the electronic address and/or an identifier 112 to indicate that the medical device 102 is assigned to or otherwise providing treatment for the patient. The patient may register using the software application 146, for example. It should be appreciated that the phone number provided by the patient should match the number provided by the clinician to enable the medical device pairing to proceed.

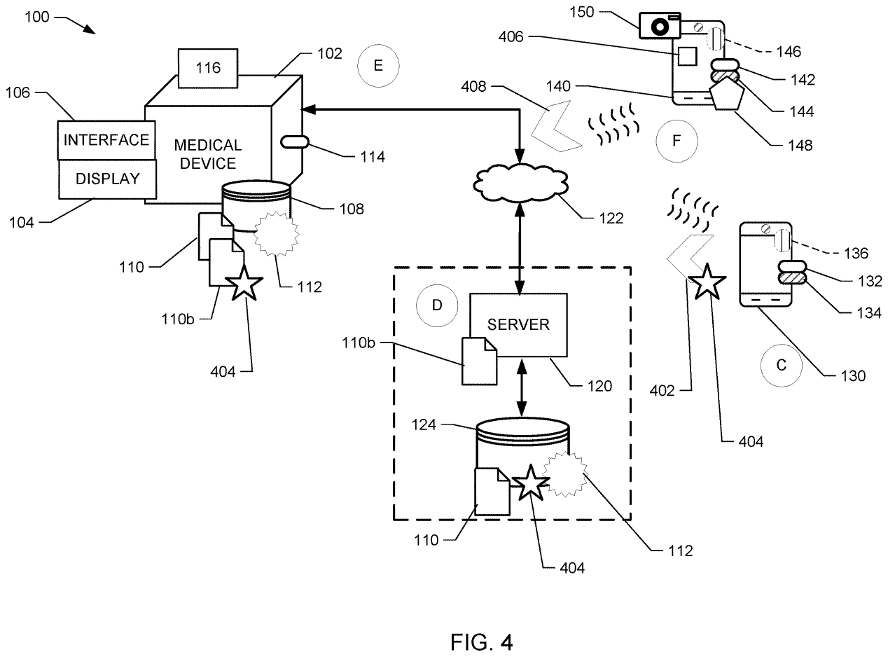

At Event C in FIG. 4, the clinician uses the software application 136 to transmit a patient device authorization message 402 to the server 120. The message 402 is indicative that the patient is authorized to pair the mobile device 140 with the medical device 102. The message 402 may include a phone number 404 of the mobile device 140. At Event D, the server 120 stores the authorization to pair and/or the phone number 404 to the memory device 124 in association with a record related to the patient. Also at Event D, the server generates a patient certificate 110b for secure communication between the medical device 102 and the mobile device 140 associated with the phone number 404. The server 120 creates the patient certificate 110b after receiving the authorization to pair from the clinician device 130.

At Event E, the server 120 transmits the patient certificate 110b and the phone number 404 to the medical device 102, which is stored to the memory device 108. The server 120 uses the secure communication connection provided, for example, by the certificate 110 to transmit the patient certificate 110 b and the phone number 404. The secure communication prevents the patient certificate 110 b and the phone number 404 from unauthorized access across the public network 122. In other embodiments, the patient certificate 110b is not created and transmitted to the medical device 102 until a request is received from the patient to pair. The request may be provided through the control interface 106 of the medical device 102 through the patient software application 146 of the mobile device 140.

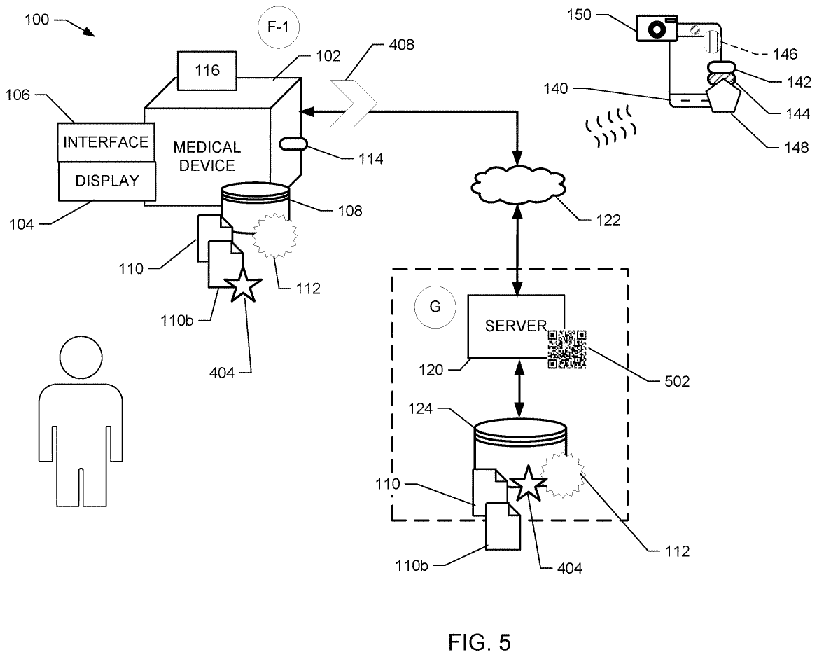

At Event F, the patient selects a pairing input 406 of the software application 146 to request to pair the mobile device 140 with the medical device 102. In other embodiments (shown as Event F-1 in FIG. 5), the patient may request the pairing using the control interface 106 of the medical device 102. In either instance, a pairing request message 408 is transmitted from the mobile device 140 (or the medial device 102) to the server 120 to indicate the patient has requested to pair the devices.

As shown in FIG. 5 at event G, after receiving the pairing request message 408 from the medical device 102 or the mobile device 140, the server 120 is configured to generate a pairing code 502. As disclosed herein, the paring code may include a QR code, a bar code, alphanumeric characters, or any other code graphical or textual code. The server 120 is configured to generate a different pairing code when the pairing request message 408 is received. Thus, different pairing codes are generated for different pairings between medical devices and mobile devices. Further, the server 120 may provide a timeout with the pairing code so that it becomes invalid after, for example, five minutes, ten minutes, etc.

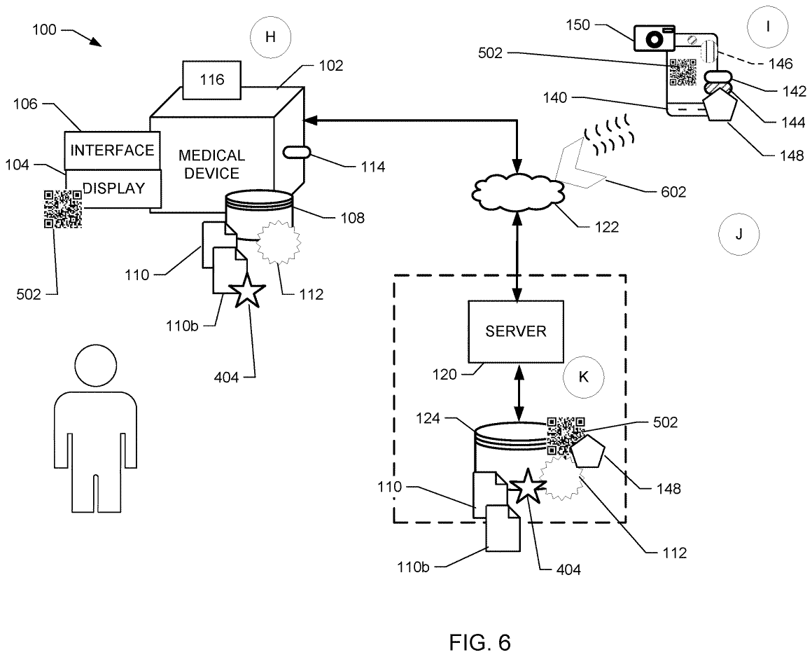

At Event H shown in FIG. 6, the server 120 transmits the pairing code 502 to the medical device 102 within one or more messages. The server 120 uses the secure communication channel with the medical device 102 for transmitting the pairing code 502 over the public network 122, thereby protecting the pairing code 502 from unauthorized access. The medical device 102 then displays the pairing code 502 via the display device 104. As shown in FIG. 6, the server 120 may also store a copy of the pairing code 502 within the memory device 124. The pairing code 502 may be stored in association with the medical device identifier 112, the certificates 110 and 110b, the phone number 404 of the mobile device 140, an EMEI of the mobile device 140 (or other device identifier 148), and/or a patient identifier.

At Event I, the mobile device 140 is used to record the pairing code 502 shown on the display device 104 of the medical device 102. In some embodiments, the camera 150 records one or more images of the pairing code 502, which causes the processor 142 to convert the QR or barcode into alphanumeric characters. The software application 146 may display a prompt for the patient to use the camera 150 to record the image of the pairing code 502. The prompt may include an example picture of a pairing code or an animation/graphic showing how the pairing code is to be recorded using the mobile device 140. The prompt may be display after the patient selects the pairing input 406.

In other embodiments, the software application 146 prompts the patient to enter alphanumeric characters of the pairing code 502 shown on the display device 104. At Event J, the software application 146 transmits a message 602 to the server 120 including at least the pairing code 502 or alphanumeric characters associated with the pairing code 502. In some embodiments, the message 602 may include the phone number 404 and/or the device identifier 148 of the mobile device 140.

At Event K, the server 120 receives the message 602. The server 602 uses the phone number 404 and/or the device identifier 148 to identify the associated record, file, or index within the memory device 124. The server 120 then performs a congruency check to ensure the pairing code 502 received from the mobile device 140 matches the pairing code 502 that was generated for the medical device 102. The server 120 may also confirm that an authorization pairing indication was received from the clinician device 130 for the mobile device 140.

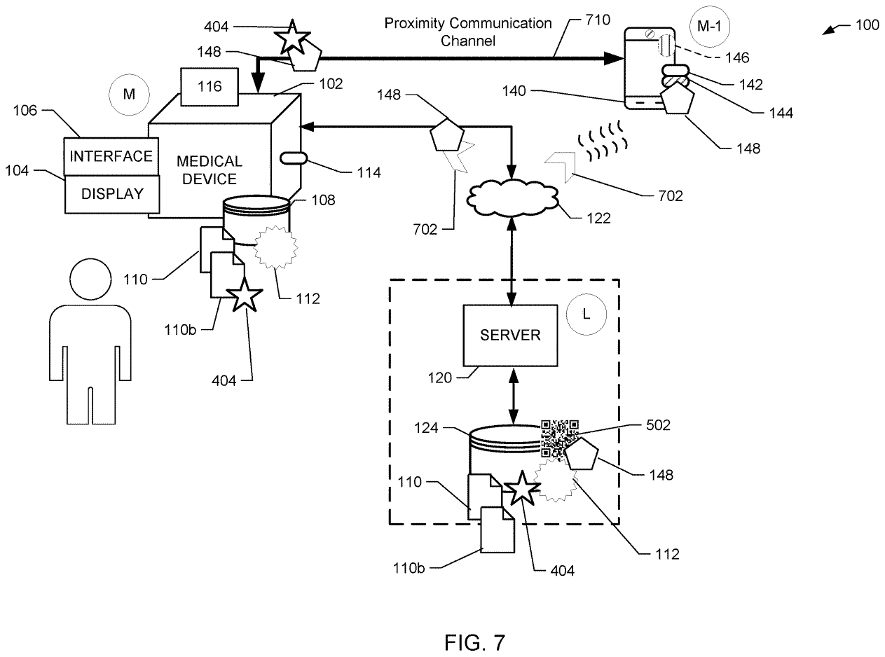

As shown on FIG. 7 at Event L, when the congruency check returns a match, the server 120 is configured to transmit a pairing acceptance message 702 to at least the medical device 102. The pairing acceptance message 702 may include the device identifier 148. At Event M, the medical device 102 receives the message 702 and opens a proximity communication channel 710 with the mobile device 140. The medical device 102 may use the device identifier 148 and/or the phone number 404 to establish the proximity communication channel 710 with the mobile device 140, which may be compliant with a Bluetooth® protocol, a Wi-Fi direct protocol, a local Wi-Fi protocol, a near-field communication (“NFC”) protocol, a Zigbee® protocol, a LoRa protocol, a Z-Wave® protocol, a WeMo® protocol, or a low-power wide-area network (“LPWAN”) protocol. To open the proximity communication channel 710, the processor 114 is configured to activate a transceiver associated with the protocol and use the device identifier 148 and/or the phone number 404 to locate and communicate with the mobile device 140. The medical device 102 may perform a pairing routine that is associated with the communication protocol used.

In some embodiments, at Event M-1, the server 120 may also transmit the pairing acceptance message 702 to the mobile device 140. The pairing acceptance message 702 may cause the mobile device 140 to activate a transceiver associated with the communication protocol to be used. For instance, the message 702 may cause the processor 142 to activate a Bluetooth® transceiver to enable the proximity communication channel 710 to be established.

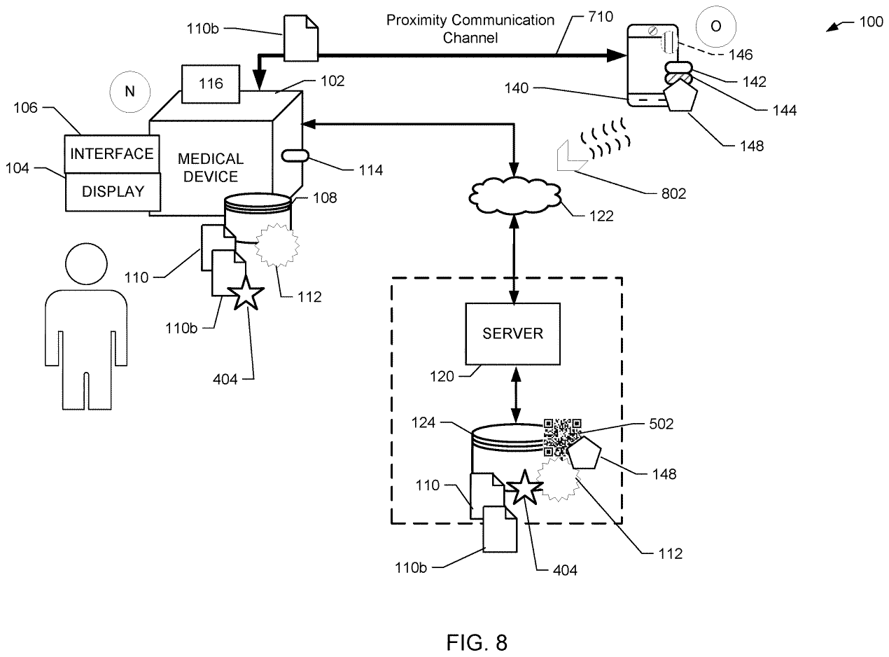

At Event N shown in FIG. 8, the medical device 102 is configured to transmit the patient certificate 110b to the mobile device 102 using the newly established proximity communication channel 710. As discussed above, the medical device 102 previously received the patient certificate 110b from the server 102 when the clinician device 130 authorized the pairing with the mobile device 140 and/or after the pairing request message 408 was received. At Event O, the mobile device 140 stores the patient certificate 110b, which enables the mobile device 140 and the medical device 102 to securely communicate with each other over the proximity communication channel 710. In some embodiments, the software application 146 transmits a pairing complete message 802 to the server 120 to indicate that the medical device 102 and the mobile device 140 were successfully paired. The server 120 may use the pairing complete message 802 to retire or expire the pairing code 502 and update a patient's EMR or other file to indicate the successful pairing. In some instances, the medical device 102 instead transmits the pairing complete message 802.

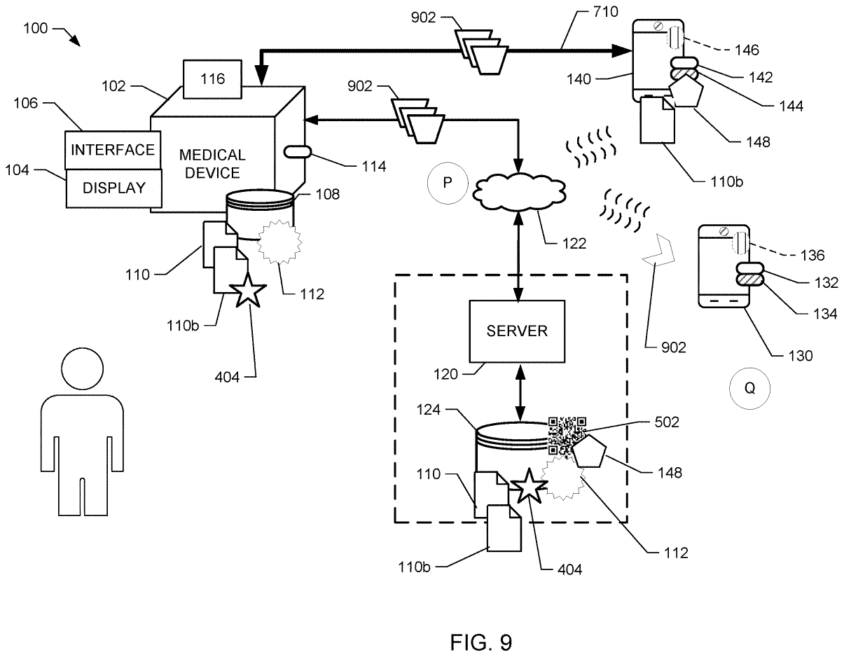

At Event P shown in FIG. 9, the medical device 102 transmits medical data 902 to the mobile device 140 using the proximity communication channel 710. The software application 146 of the mobile device 140 may use the proximity communication channel 710 to transmit operating instructions to the medical device 102. Further, the medical device 102 uses the secure connection with the server 102 to transmit the medical data 102 for storage in the patient's EMR within the memory device 124. At this point, the mobile device 140 has successfully paired with the medical device 102 using a secure process that safeguards patient data while at the same time only requires a patient to record a pairing code displayed by the medical device 102.

In some embodiments, shown at Event Q, the server 120 may transmit a pairing complete message 902 to the clinician device 130. The pairing complete message 902 informs a clinician that the patient was able to successfully pair the mobile device 140 with the medical device 102. When the pairing is not successful, the server 102 may transmit a message indicative of the failure to pair. A clinician (or staff associated with the clinician) may use the failure message as a prompt to reach out to the patient to help them with the pairing process. The server 102 may generate the failure message when the congruency check does not produce a match or the pairing code 502 is not received from the mobile device 140 within a defined timeout period.

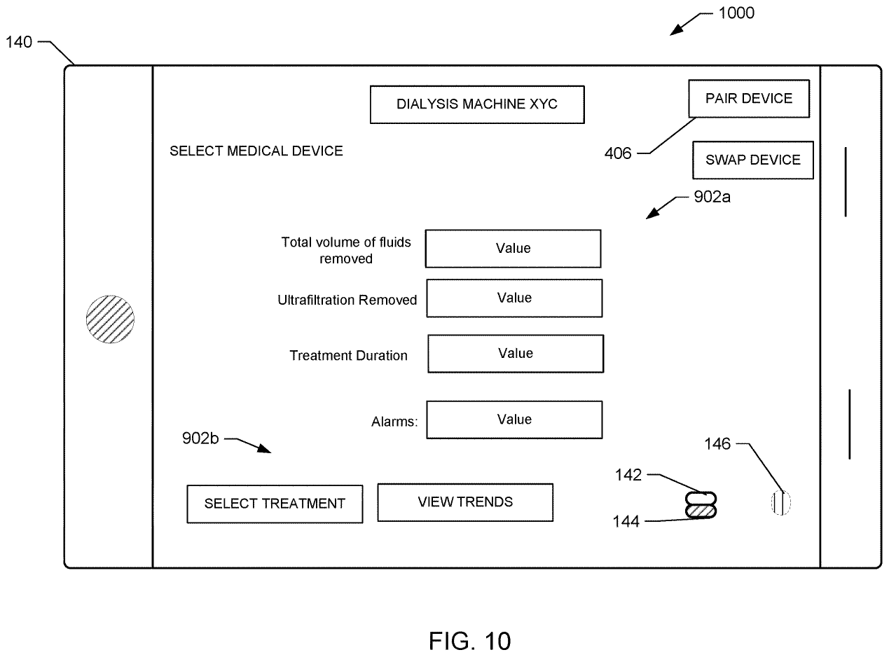

FIG. 10 is a diagram of an example user interface 1000 provided by the patient software application 146 of the mobile device 140, according to an example embodiment of the present disclosure. The user interface 1000 includes a pairing input 406 to enable the patient to pair the mobile device 140 with the medical device 102. In this instance, the mobile device 140 has already been paired, as shown by the display of the medical device identifier 112 (i.e., Dialysis Machine XYC).

The user interface 1000 includes medical data 902a that was received from the medical device 102 via the secure proximity communication channel 710. The user interface 1000 also includes options to change prescriptions or prescription parameters 902b for a dialysis treatment. Together, the medical data 902a provides a patient with information regarding how their dialysis treatment is progressing and trending overtime. Further, the medical data 902a gives a patient at least some control over which treatment is to be performed to better coincide with their lifestyle. The use of the proximity communication channel 710 ensures the medical data 902 is not exposed in an unsecure manner across the public network 122 and prevents a malicious application from having access to and affecting a treatment performed by the medical device 102.

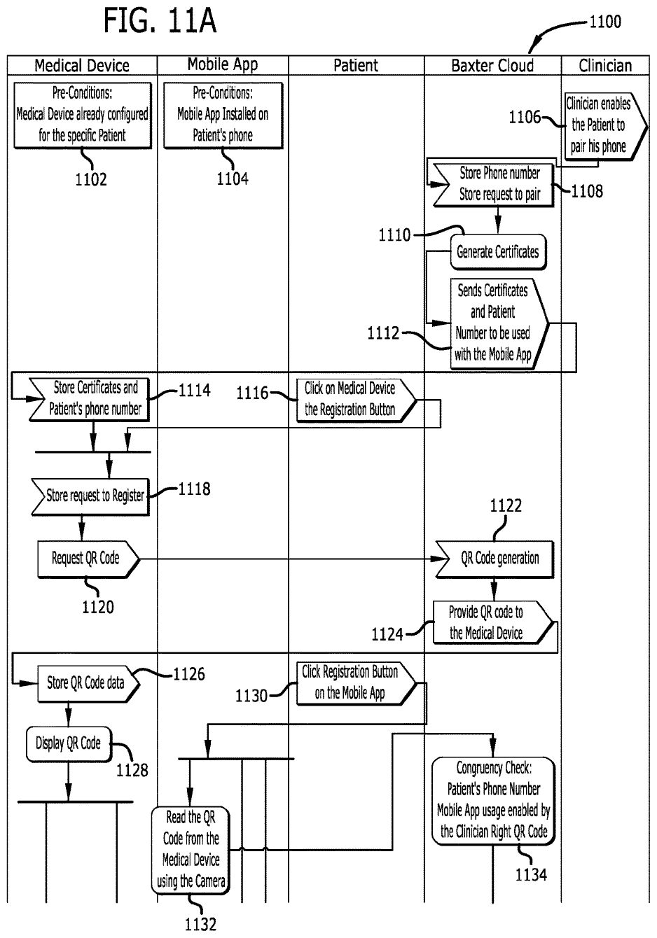

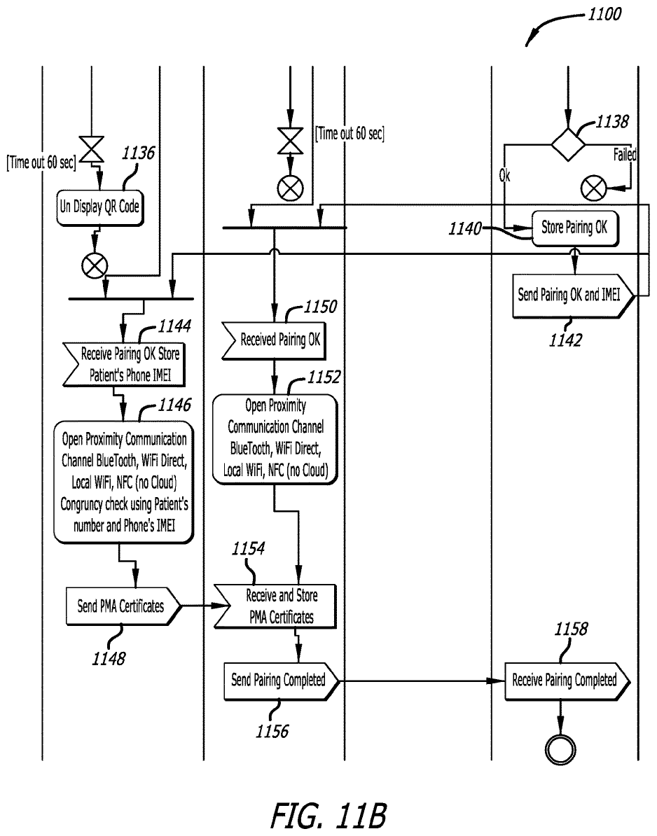

FIGS. 11A and 11B show flow diagrams of an example procedure 1100 for pairing a medical device with a mobile device, according to an example embodiment of the present disclosure. Although the procedure 1100 is described with reference to the flow diagram illustrated in FIGS. 11A and 11B, it should be appreciated that many other methods of performing the steps associated with the procedure 1100 may be used. For example, the order of many of the blocks may be changed, certain blocks may be combined with other blocks, and many of the blocks described may be optional. The operations described in the procedure 1100 are specified by one or more instructions and may be performed among multiple devices including, for example, the server 120, the medical device 102, the clinician device 130, and/or the patient mobile device 140.

The example procedure 1100 begins when the medical device 102 is configured for a specific patient (block 1102). This may include programming the medical device 102 with an identifier of the patient. This may also include storing at the memory device 124 via the server 120, a record (such as an EMR) that associates an identifier of the patient with an identifier or address of the medical device 102. This may also include installing one or more certificates on the medical device 102 to enable secure communication across the public network 122. Additionally, the patient software application 146 (block 1104) is installed on the patient mobile device 140. Installation may include registering the mobile device 140 with the server 120 or associating a phone number of the mobile device 140 with the patient's record.

Later, a clinician uses the mobile application 136 to authorize the patient to pair their mobile device 140 with the medical device 102 (block 1106). As described above in connection with FIGS. 1 to 3, this may include selecting a patient (e.g., a patient identifier), selecting a medical device identifier, and/or selecting or providing a phone number of the mobile device 140. The software application 136 is configured to receive and transmit the selections to the server 120, which stores the phone number and the pairing authorization in association with the patient record (block 1108).

The server 102 also generates one or more patient certificates (e.g., the certificate 110b discussed in connection with FIGS. 4 to 9) after receiving the authorization from the clinician device 130 (block 1110). The server 120 transmits the one or more patient certificates 110b to the medical device 102 in association with the phone number of the mobile device 140 that is to be paired (block 1112). The medical device 102 receives and stores the certificate 110b and the phone number (block 1114).

In some embodiments, the patient uses the control interface 106 of the medical device 102 to select an option to register and/or pair with the mobile device (block 1116). The medical device 102 stores the request to register or pair (block 1118) and transmits a message to the server 120 to request a pairing code (block 1120). The server 120 receives the request and generates a pairing code (block 1122). In some instances, the server 120 uses a random number/character generator to generate the pairing code. The server 120 then transmits the generated pairing code to the medical device 102 (block 1124), which stores the pairing code (block 1126). The medical device 102 then displays the pairing code on a display screen (block 1128). In some instances, the pairing code is displayed for a defined time period.

During this time, the patient may select a pairing request input shown by the patient software application 146 on the mobile device 140 (block 1130). Selection of this input causes the software application 146 to display a prompt to record the pairing code (block 1132). This may include opening a camera application and enabling the patient to record one or more images of the pairing code displayed by the medical device 102. This may also include displaying one or more data fields for receiving a text input of the pairing code. The software application 146 transmits the pairing code to the server 102, which performs a congruency check (block 1134). As discussed above, this check includes comparing the received pairing code and related phone number from the mobile device 140 with the generated pairing code that is stored in association with a phone number of the mobile device 140 within an EMR or other patient record. The congruency check may also include ensuring the patient record includes an indication from a clinician indicating the pairing authorization, including a phone number of the mobile device 140 for pairing.

Turning to FIG. 11B, the example procedure 1100 continues as the server 120 performs the congruency check. The medical device 102 may remove the display of the pairing code after an elapsed time has approached (block 1136). When the congruency check is successful (block 1138), the server 120 stores to the patient record an indication that the pairing is approved (block 1140). The server 120 then transmits a message to the medical device 102 that is indicative of the pairing acceptance and a device identifier (such as the IMEI number) of the mobile device 140 (block 1142). When the congruency check is not successful, the server 120 may send a failure message to at least one of the medical device 102 for display on a screen, the software application 146 of the mobile device 140 and/or the clinician device 130.

As shown in FIG. 11B, the medical device 102 receives the message indicative of the successful congruency check from the server 120 (block 1144). The medical device 102 then uses the mobile device identifier and phone number to open a proximity communication channel with the mobile device (block 1146). After the channel is open, the medical device transmits the patient certificate 110b (block 1148) to complete the pairing process and enable secure communication with the mobile device 140. In some embodiments, the mobile device 140 also receives the message indicative that the congruency check was successful (block 1150). In response, the mobile device 140 opens the proximity communication channel to enable the protocol connection requests from the medical device 102 to be received (block 1152). After the channel is open, the mobile device 140 receives and stores the patient certificate (block 1154).

After the proximity communication channel is established, the medical device 102 and/or the mobile device 140 transmits a pairing complete message to the server (block 1156). The server 120 receives the message and stores an indication of the pairing to the patient's record (block 1158). The server 120 may also transmit a message to the clinician device 130 to indicate the successful pairing. The example procedure 1100 then ends until another pairing procedure is initiated. As one can appreciate, the example procedure 1100 can be carried out quickly with minimal input from a patient. Namely, the patient only needs to request to obtain a pairing code and record the pairing code using a mobile device. The server 120 and the medical device 102 are configured to communicate over a secure communication channel to facilitate the pairing with the mobile device 140. The example procedure is accordingly efficient and secure in compliance with multifactor authentication.

In some embodiments, the medical device 102 and the server 120 are configured to enable a patient and/or a clinician to cancel the pairing. In one example, the control interface 106 receives a request from the patient to un-pair the medical device 102 from the mobile device 140. In response, the medical device 102 transmits an un-pair request message to the server 120, which causes the server 120 update a patient record stored in the memory device 124 to remove the pairing. The server 120 may also send a confirmation message to the medical device 102, which causes the medical device 102 to remove the patient certificate 110b and/or phone number of the mobile device 140. A clinician may also initiate the un-pairing process by sending a message from the software application 136 to the server 120 requesting to remove the pairing. A clinician may also change the patient's phone number, which removes the pairing but enables the patient to pair the medical device 102 with a new mobile device.

In some embodiments, the server 120 is further configured to enable a patient to swap medical devices 102. The patient software application 146 may include an option to initiate a swap, as shown in FIG. 10. After selecting the swap option, the software application 146 may prompt the patient for a code and/or biometric data. After authenticating the patient, the software application 146 transmits a swap request message to the server 120, which performs a new congruency check for a new medical device assigned to the patient. When the congruency check is successful, the server 120 enables the new medical device 102 to pair with the mobile device 140, as discussed above.

### III. Conclusion

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A system for pairing a patient device with a medical device, the system comprising:
a medical device configured to perform a treatment or measurement on a patient, the medical device communicatively coupled to a network;
a database configured to correlate a patient phone number and a device identifier with an identifier, an electronic address, or a device identifier of the medical device;
a server communicatively coupled to the database and the network, the server securely connected to the medical device via a mutual authentication mechanism, the server configured to:
generate a patient certificate and transmit the patient certificate and the patient phone number to the medical device to enable pairing,
generate a pairing code after receiving a code request message,
store to the database an association between the pairing code and the patient phone number, and
transmit the pairing code to the medical device, causing a display screen of the medical device to display the pairing code;
a patient device communicatively coupled to the network, the patient device including a processor and a memory device storing one or more instructions defining an application for communicating with the medical device, execution of the one or more instructions by the processor causing the application on the patient device to at least:
receive a selection of a pairing input,
after receiving the pairing input, enable the patient device to record information indicative of the pairing code displayed by the medical device,
transmit the pairing code and the patient phone number to the server causing the server to perform a congruency check by comparing the received pairing code and the patient phone number to the pairing code and the patient phone number stored in the database, and
when there is a match between the received pairing code and the patient phone number and the pairing code and the patient phone number stored in the database, receive from the server a pairing acceptance message that causes the patient device to open a proximity communication channel,
wherein the server is configured to transmit the pairing acceptable message and the device identifier of the patient device to the medical device when there is a match between the received pairing code and the patient phone number and the pairing code and the patient phone number stored in the database,
wherein the medical device is configured to open the proximity communication channel using the patient phone number and the device identifier to communicate with the patient device, and
wherein the medical device transmits the patient certificate to the application of the patient device using the opened communication channel, enabling the application to communicate with the medical device and the server via the network.

2. The system of claim 1, wherein the server is configured to receive from a clinician device a patient device enable message to permit the patient to pair the patient device using the application.

3. The system of claim 2, wherein, after receiving the patient device enable message, the server is configured to generate the patient certificate.

4. The system of claim 3, wherein the server additionally causes the application to enable selection of the pairing input after receiving the patient device enable message.

5. The system of claim 2, wherein the database receives at least one of the patient phone number or the device identifier from the clinician device via the patient device enable message.

6. The system of claim 2, wherein the application on the patient device is further configured to transmit a pairing completed message to the server, enabling the server to display an indication to the clinician device indicative of the pairing of the patient device.

7. The system of claim 1, wherein the application is configured to transmit at least one of the patient phone number or the device identifier to the database after the application is installed on the patient device.

8. The system of claim 1, wherein the application is configured to transmit the device identifier of the patient device after receiving the pairing acceptance message from the server.

9. The system of claim 1, wherein the patient certificate includes a parts manufacturer approval (“PMA”) certificate.

10. The system of claim 1, wherein the device identifier includes at least one of a media access control (“MAC”) address or an International Mobile Equipment Identity (“IMEI”) number.

11. The system of claim 1, wherein the proximity communication channel encompasses at least one of a Bluetooth® protocol, a Wi-Fi direct protocol, a local Wi-Fi protocol, a near-field communication (“NFC”) protocol, a Zigbee® protocol, a LoRa protocol, a Z-Wave® protocol, a WeMo® protocol, or a low-power wide-area network (“LPWAN”) protocol, and
wherein the network includes at least one of a cellular network or a wide area network.

12. The system of claim 1, wherein the medical device includes a user interface configured to receive a pairing request input, and wherein the medical device transmits the code request message to the server after receiving the pairing request input.

13. The system of claim 1, wherein the pairing code includes at least one of a Quick-Response (“QR”) code, a barcode, or an alpha-numeric code.

14. The system of claim 1, wherein the database is configured to correlate the patient phone number or identifier based on a clinician request.

15. The system of claim 1, wherein the server and the medical device each include a copy of a same certificate to communicate via the mutual authentication mechanism.

16. (canceled)

17. The system of claim 1, wherein the application on the patient device, the server, and the medical device are configured to use the patient certificate to encrypt communications therebetween.

18. (canceled)

19. The system of claim 1, wherein the server also performs the congruency check by confirming a clinician of the patient provided an indication that the patient is permitted to pair the patient device with the medical device.

20. The system of claim 1, wherein the information indicative of the pairing code is obtained via an image recorded by a camera of the patient device.

21. An apparatus for pairing a patient device with a medical device, the apparatus comprising:
a database configured to correlate a patient phone number and a device identifier with an identifier, an electronic address, or a device identifier of a medical device that is configured to perform a treatment or a measurement on a patient; and
a server communicatively coupled to the database and securely connected to the medical device via a mutual authentication mechanism over a network, the server configured to:
generate a patient certificate and transmit the patient certificate and the patient phone number to the medical device to enable pairing,
generate a pairing code after receiving a code request message from the medical device,
store to the database an association between the pairing code and the patient phone number,
transmit the pairing code to the medical device, enabling a display screen of the medical device to display the pairing code,
receive, from an application operating on a patient device communicatively coupled to the network, the pairing code and the patient phone number as deciphered from an image recorded by a camera of the patient device of the display screen of the medical device,
perform a congruency check by comparing the received pairing code and the patient phone number to the pairing code and the patient phone number stored in the database, and
when there is a match between the received pairing code and the patient phone number and the pairing code and the patient phone number stored in the database:
transmit to the patient device a pairing acceptance message that causes the patient device to open a proximity communication channel, and
transmit the pairing acceptable message and the device identifier of the patient device to the medical device, causing the medical device to open the proximity communication channel using the patient phone number and the device identifier to communicate with the patient device,
wherein the medical device transmits the patient certificate to the application of the patient device using the opened communication channel, enabling the application to communicate with the medical device and the server via the network.

22. A medical device apparatus for pairing with a patient device, the medical device apparatus comprising:
a memory device configured to store at least a pairing code, a patient phone number, a device identifier, and a patient certificarte;
a user interface;
a display screen; and
a processor communicatively coupled to the memory device, the display screen, and the user interface, the processor and memory device configured to cooperatively:
receive, from a server via a network, the patient certificate and the patient phone number, wherein the processor is authenticated to communicate with the server,
store, to the memory device, the patient certificate and the patient phone number,
receive, via the user interface, a request to pair with a patient device,
transmit, to the server, a request for a pairing code,
receive, from the server, the pairing code,
display, via the display screen, the pairing code to enable a camera of the patient device to record an image of the pairing code,
receive, from the server, a pairing acceptable message and the device identifier of the patient device when there is a match between the pairing code and the patient phone number received in the server from the patient device and the pairing code and the patient phone number stored in a database,
open a proximity communication channel using the patient phone number and the device identifier to communicate with the patient device, and
transmit the patient certificate to the application of the patient device using the opened communication channel, enabling the processor to communicate with the application of the patient device.

23. (canceled)
